(19) United States
(12) Patent Application Publication
(10) Pub. No.: US 2020/0100661 A1
(43) Pub. Date: Apr. 2, 2020

(54) INTEGRATED ENDOSCOPE AND VISUALIZATION SYSTEM

(71) Applicants: HJY Forward Medical Investment Co., Ltd., Ebene (MU); Jorjin Technologies Inc., New Taipei City (TW); Peer Giant System Inc., New Taipei City (TW); New Ken Technologies Co.,LTD., Hsinchu (TW)

(72) Inventors: John Jiannyuh CHEN, Seattle, WA (US); Wen-Lung LIANG, New Taipei City (TW); Kwo-Hsiang CHANG, New Taipei City (TW); Pao-Chyuan CHEN, Hsinchu (TW); Shiann-Jang WANG, New Taipei City (TW); Chih-Chung HU, New Taipei City (TW)

(73) Assignees: HJY Forward Medical Investment Co., Ltd., Ebene (MU); Jorjin Technologies Inc., New Taipei City (TW); Peer Giant System Inc., New Taipei City (TW); New Ken Technologies Co.,LTD., Hsinchu (TW)

(21) Appl. No.: 16/146,402

(22) Filed: Sep. 28, 2018

(51) Int. Cl. A61B 1/05; A61B 1/00; A61B 90/00

(52) U.S. Cl. CPC A61B 1/051; A61B 1/00009; A61B 2090/364; A61B 1/00096; A61B 90/36

(57) ABSTRACT

An integrated endoscope and visualization system comprises an endoscope, a visualization processing module and a 3D display device such as smart-glasses or naked-eye 3D display. The endoscope is equipped with two image capturing modules such as CMOS image sensors and can be disposable. The visualization processing module receives and encodes the images obtained by the two image capturing modules in order to generate an output visualization signal. The output visualization signal is transmitted to and displayed by the 3D display device in a 3D manner. The integrated endoscope and visualization system not only provides a 3D vision of surgical target during an endoscopic surgery operation, but also allow the medical personnel to walk or move during the surgery while keeping looking at the 3D images captured by the endoscope through wearable display devices. In addition, the modularized visualization processing module can be readily connected with the endoscope, display devices and an IPC for further connecting to other external medical devices.

### 1. FIELD OF THE INVENTION

The invention refers to an integrated endoscope and visualization system, especially refers to an integrated endoscope and visualization system that can provide a three-dimensional (3D) vision of surgical target during an endoscopic surgery operation.

### 2. DESCRIPTION OF THE PRIOR ART

Minimally invasive surgery using endoscopic vision has become a trend in surgery because of its advantages of minimal invasion, small wound, less pain and quick recovery. Typically, resection lesions can be achieved by endoscopic surgery with several small wounds, approximately one centimeter long. Therefore, wound pain of the patients is relieved by reducing the wound area. The recovery time and the number of hospitalization days are shortened.

Generally speaking, an endoscopic surgery system comprises an endoscope, a display device for showing the image acquired by the endoscope, a processor for managing image signals, and surgery tools and instruments for operating the surgery. However, conventional endoscopic surgery system has the following deficiencies and thus is required for further improvements: (1) the endoscope includes a prismatic light guide apparatus to guide image light from its front tip to the image capturing sensor located at the rear end of endoscope, the prismatic light guide apparatus is complex in structure and is expensive; (2) the endoscope uses a charge-coupled device (CCD) to be the image capturing sensor, and thus requires additional analog-to-digital (A/D) converter to transform analog signals of CCD into digital signals, the A/D converter will cause image distortion; (3) due to high cost, the endoscope is non-disposable and thus results in possible infection even though the endoscope is sterilized after use; (4) the display is a stationary equipment fixed at a predefined location, the medical personnel cannot walk or move freely with real-time visualization; (5) the image of three-dimensional (3D) tissues or organs is captured by the endoscope and shown on the display in a two-dimensional (2D) manner, and thus makes it more difficult for the medical personnel to positioning real points of the captured image while conducting the surgery.

### SUMMARY OF THE INVENTION

Accordingly, the primary objective of the present invention is to provide an integrated endoscope and visualization system, which can provide a 3D vision of surgical target during an endoscopic surgery operation.

Another objective of the present invention is to provide an integrated endoscope and visualization system which includes wearable display devices, such that the medical personnel is free to walk or move during the surgery while keeping looking at the images captured by the endoscope through the wearable display devices.

Yet another objective of the present invention is to provide an integrated endoscope and visualization system which includes a modularized visualization processing module that can be readily connected with the endoscope, display devices and an industrial personal computer (IPC) for further connecting to other external medical devices.

A further objective of the present invention is to provide an integrated endoscope and visualization system which includes an endoscope that is relatively simpler in structure and cheaper in cost, and thus can be disposable.

In order to achieve aforementioned objectives, the present invention discloses an integrated endoscope and visualization system, comprising:

an endoscope, equipped with a first image capturing module, a second image capturing module, and a cable for electrically connecting said first image capturing module and said second image capturing module; said first image capturing module being capable of capturing a first image and transforming said first image into a first electronic signal; said second image capturing module being capable of capturing a second image and transforming said second image into a second electronic signal;

a visualization processing module, connected with the endoscope; said visualization processing module receiving said first electronic signal and said second electronic signal in order to generate an output visualization signal containing the first image and the second image; and

at least one 3D display device, for receiving said output visualization signal and displaying said first image and said second image in a 3D manner.

In a preferred embodiment, the first image capturing module and the second image capturing module are arranged in a side-by-side configuration and each comprises a lens set and a CMOS image sensor or CCD image sensor.

In a preferred embodiment, the 3D display device is one of the following: smart-glasses, naked-eye 3D display.

In a preferred embodiment, the smart-glasses comprises a left eye display and a right eye display for displaying the first image and the second image respectively in an Augmented Reality (AR) manner.

In a preferred embodiment, the visualization processing module comprises: an input unit communicated with the endoscope either in a wired or wireless manner for receiving said first electronic signal and said second electronic signal, a control unit connected with the input unit for processing received said first electronic signal and said second electronic signal in order to generate said output visualization signal, a memory unit for pre-storing an application for driving the control unit and caching said output visualization signal, and a connecting interface unit for communicating with at least the 3D display device.

In a preferred embodiment, the visualization processing module communicates with the 3D display device by using one of the following interfaces: USB, HDMI, Wi-Fi, LAN, Airplay, Miracast, Bluetooth.

In a preferred embodiment, the integrated endoscope and visualization system further comprises an industrial personal computer (IPC) connected with the visualization processing module.

In a preferred embodiment, the endoscope is disposable and sterilizable, and can be detached from the visualization processing module.

In a preferred embodiment, the visualization processing module is a modularized component and is connected to the IPC in a detachable manner.

### BRIEF DESCRIPTION OF THE DRAWINGS

The present invention will now be specified with reference to its preferred embodiment illustrated in the drawings, in which:

FIG. 1 is a schematic view of an embodiment of the integrated endoscope and visualization system in accordance with the present invention;

FIG. 2 is a schematic drawing showing a side view and a front end view of an embodiment of the endoscope in accordance with the present invention;

FIG. 3 is a block diagram schematically showing an architecture of the integrated endoscope and visualization system in accordance with the present invention;

FIG. 4 is a schematic view of the smart-glasses in accordance with the present invention; and

FIG. 5 is a block diagram of the smart-glasses in accordance with the present invention.

### DESCRIPTION OF THE PREFERRED EMBODIMENT

The invention disclosed herein is directed to an integrated endoscope and visualization system that comprises an endoscope, a visualization processing module and a 3D display device such as smart-glasses or naked-eye 3D display. The endoscope is equipped with two image capturing modules such as CMOS image sensors or CCD image sensors and can be disposable. The visualization processing module receives and encodes the images obtained by the two image capturing modules in order to generate an output visualization signal. The output visualization signal is transmitted to and displayed by the 3D display device in a 3D manner. The integrated endoscope and visualization system not only provides a 3D vision of surgical target during an endoscopic surgery operation, but also allow the medical personnel to walk or move during the surgery while keeping looking at the 3D images captured by the endoscope through wearable display devices. In addition, the modularized visualization processing module can be readily connected with the endoscope, display devices and an IPC for further connecting to other external medical devices.

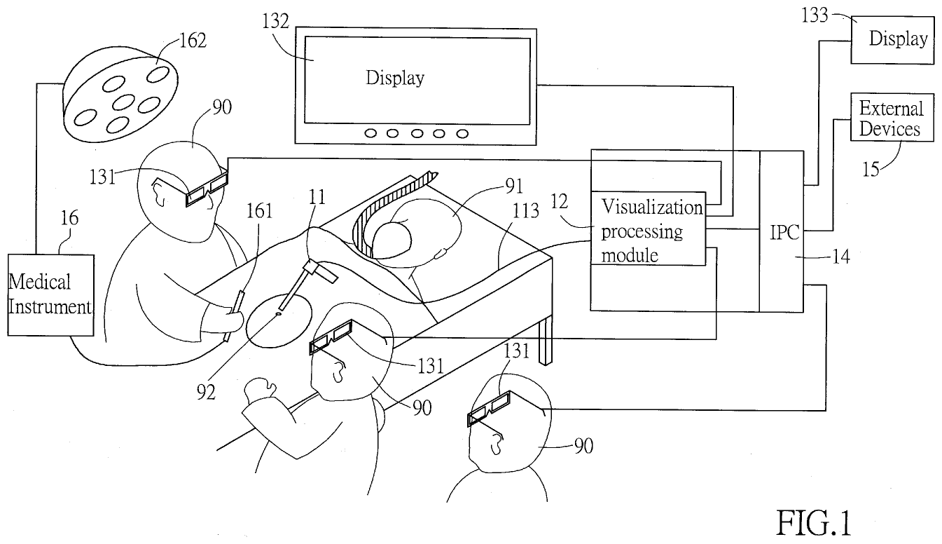

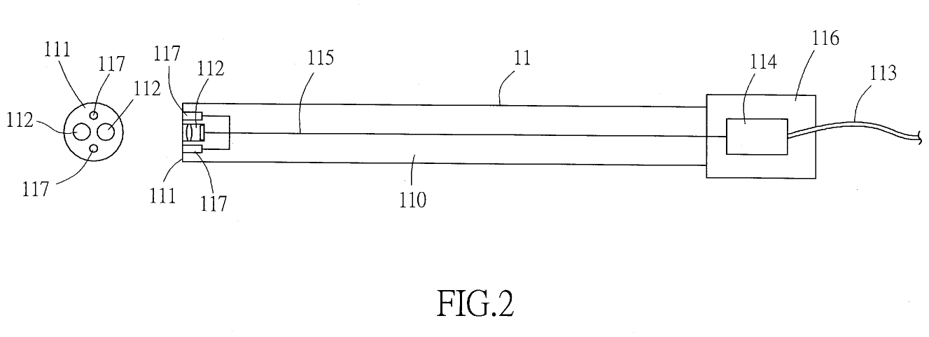

Please refer to FIG. 1, which is a schematic view of an embodiment of the integrated endoscope and visualization system in accordance with the present invention. The integrated endoscope and visualization system is suitable for performing minimally invasive endoscopic surgeries. In this embodiment, the integrated endoscope and visualization system comprises: an endoscope 11, a visualization processing module 12 and at least one 3D display device 131, 132, 133. The endoscope 11 is operated by medical personnel 90 to approach a surgery target of a patient 91 through a small wound 92. The endoscope 11 is equipped with a first image capturing module 112 (please also refer to FIG. 2), a second image capturing module 112, and a cable 113 for electrically connecting the first and second image capturing modules 112. The endoscope 11 is connected to the visualization processing module 12 via the cable 113, such that the visualization processing module 12 is able to receive and encode the images obtained by the first and second image capturing modules 112 in order to generate an output visualization signal containing 3D video streaming. The 3D display devices 131, 132, 133 are communicating with the visualization processing module in a wired or wireless manner. The 3D display device 131, 132, 133 receives the output visualization signal and displays the 3D video streaming in a 3D manner. In this embodiment, the 3D display device 131, 132, 133 can be a smart-glasses 131 or a naked-eye 3D display 132, 133. When the 3D display device 131 is the smart-glasses, the medical personnel 90 can wear such smart-glasses and watch the images acquired by the first and second image capturing modules 112 of endoscope 11 in a 3D and yet Augmented Reality (AR) manner. When the 3D display device 132, 133 is the naked-eye 3D display, it can be fixed on the wall or other places for other medical personnel 90 to watch with bare-eyes without wearing the smart-glasses. In another embodiment, one or more display device 132, 133 can also be the ordinary 2D display monitors. By watching the 3D images of the surgery target shown on either the smart-glasses or the naked-eye 3D display, the medical personnel 90 can easily, steadily and precisely operate the medical tool 161 and/or medical instruments 16 to perform the minimally invasive endoscopic surgery. In this embodiment, the integrated endoscope and visualization system further comprises an industrial personal computer (IPC) 14 connected with the visualization processing module 12. The IPC 14 is able to be connected with other external devices 15 or other display devices 133. The external devices 15 can be ordinary or commonly used medical instruments, such as, but not limited to, surgical lamp machine 162, electrocardiographic machine, bipolar radio-frequency (RF) machine, cold light providing machine, power providing machine, etc.

Please refer to FIG. 2, which is a schematic view showing a side view and a front end view of an embodiment of the endoscope in accordance with the present invention. The endoscope 11 has an elongated thin-tube body 110 and is equipped with a first image capturing module 112, a second image capturing module 112, and a cable 113,115 for electrically connecting the first and second image capturing modules 112. The first and second image capturing modules 112 are furnished on the front tip 111 of the thin-tube body 110. A front portion of the cable 115 is received inside the thin-tube body 110, having one end thereof connected to both the first and second image capturing modules 112, while the other end is connected to a printed circuit board (PCB) 114 located at the rear part 116 of the thin-tube body 110. A rear portion of the cable 113 is connected to the PCB 114 and extends outside of the endoscope 11. In a preferred embodiment, the PCB 114 includes a socket for connecting with the rear portion of the cable 113 in a detachable manner. The endoscope 11 is connected to the visualization processing module 12 via the rear portion of the cable 113, such that the visualization processing module 12 is able to receive and encode the images obtained by the first and second image capturing modules 112 in order to generate an output visualization signal containing 3D video streaming. In this embodiment, the endoscope 11 is disposable and can be detached from the visualization processing module 12.

In a preferred embodiment, the first image capturing module 112 and the second image capturing module 112 are arranged in a side-by-side configuration and each comprises a lens set and a complementary metal oxide semiconductor (CMOS) image sensor. However, in another embodiment, these two CMOS image sensors can also be replaced by CCD image sensors. The first image capturing module 112 is capable of capturing a first image and transforming said first image into a first electronic signal. The second image capturing module 112 is capable of capturing a second image and transforming said second image into a second electronic signal. When the first and second image capturing modules 112 are both CMOS image sensors, the first and second electronic signals are digital signals, and therefore, there is no A/D converter on the PCB 114 of the endoscope. When the first and second image capturing modules 112 are both CCD image sensors, A/D converter is needed on the PCB 114 of the endoscope. In another preferred embodiment of the invention, the front tip 111 of endoscope 11 is further equipped with one or two light sources 117, such as, but not limited to, an optical fibers or LEDs, in order to emit lights toward the surgery target.

In this embodiment, the lens set can include glass lenses, plastic lenses, or PC polycarbonate lenses.

In a preferred embodiment, the endoscope 11 is further equipped with a heat dissipation element (not shown in figures) to allow temperature control at the allowable temperature range during operation (surgery). The endoscope 11 is sterilizable by gamma ray, Oxide ethylene, submerged or other sterilization procedure before use. Moreover, in addition to the two image capturing modules 112 and the light sources 117, the thin-tube body 110 of endoscope 11 can also be equipped with passages (not shown in figures) for water and surgical instruments, such that, water can pass through the passage in order to flush tissues with bleeding and pro-inflammatory substances during the surgery, while a surgical instrument such as laser knives, water jets, etc. can reach the surgical target via another passage in order to be operated under the image capturing modules 112 and the light sources 117. Furthermore, the endoscope 11 can be made of either: rigid material such as 316 stainless steel or polycarbonate, semi-rigid material (for example, the front tip 111 is made of thermal plastic urethane while the thin-tube body 110 is made of rigid material), or flexible or bendable materials, such that the endoscope 11 of the invention is suitable for all types of minimally invasive surgeries.

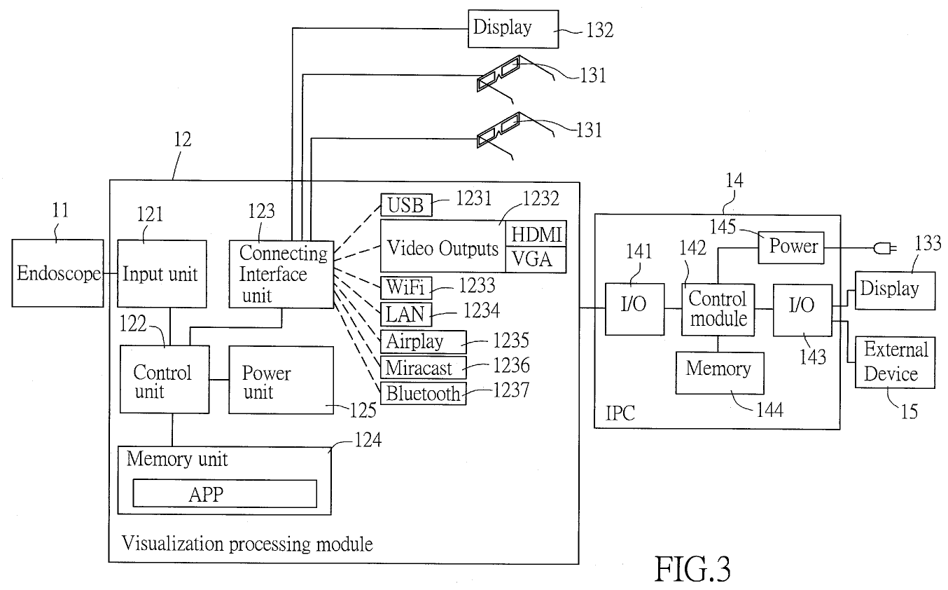

Please refer to FIG. 3, which is a block diagram schematically showing an architecture of the integrated endoscope and visualization system in accordance with the present invention. The visualization processing module 12 is connected with the endoscope 11 via the cable 113. The visualization processing module 12 receives the first electronic signal and the second electronic signal in order to generate an output visualization signal containing the first image and the second image.

In this embodiment, the visualization processing module 12 comprises: an input unit 121, a control unit 122, a connecting interface unit 123, a memory unit 124, and a power unit 125. The input unit 121 is connected with the cable 113 in a detachable manner for receiving said first electronic signal and said second electronic signal from the endoscope 11. The control unit 122 is connected with the input unit 121 for processing those received first and second electronic signals in order to generate the output visualization signal. In this embodiment, the control unit 122 contains one or more of the following electronic components: central processing unit (CPU), graphic processing unit (GPU), micro-controlling unit (MCU), micro-processing unit (MPU) and etc., so as to process and encode the received first and second electronic signals into a 3D video streaming and then output the 3D video streaming as the output visualization signal. The connecting interface unit 123 is connected to the control unit 122 and is capable of providing features for connecting and communicating with other devices such as, but not limited to, display devices 131, 132, smart-glasses, naked-eye 3D display, and IPC 14. In this embodiment, the connecting interface unit 123 supports at least some of the following standards and communication protocols: universal serial bus (USB) 1231, Video Outputs 1232 such as High Definition Multimedia Interface (HDMI) or Video Graphics Array (VGA), Wi-Fi 1233, local access network (LAN) 1234, Airplay 1235, Miracast 1236, Bluetooth 1237, and etc. The connection between the display devices 131, 132 and the connecting interface unit 123 can be either wired or wireless. In case the connection is wired, the USB 1231, Video Outputs 1232 (HDMI or VGA) or LAN 1234 of the connecting interface unit 123 can be used for connecting the display devices 131, 132, smart-glasses, naked-eye 3D display and/or IPC 14. In other case when the connection is wireless, the Wi-Fi 1233, Airplay 1235, Miracast 1236 and/or Bluetooth 1237 interfaces of the connecting interface unit 123 can be used to transmit the output visualization signal to the display devices 131, 132, smart-glasses and/or naked-eye 3D display for displaying. The memory unit 124 is for pre-storing an application (APP) for driving the control unit and caching the 3D video streaming of output visualization signal. In this embodiment, the APP provides at least the features for encoding the first and second electronic signals into the 3D video streaming and for driving the control unit 122 and connecting interface unit 123. The power unit 125 provides the electricity required by all of the components of the visualization processing module 12. In this embodiment, the power unit 125 includes a converter for converting an outside electric current to an operating electric current suitable for driving the control unit 122. In this embodiment, the visualization processing module 12 is a modularized component and is connected to the IPC 14 in a detachable manner.

In this embodiment, the IPC 14 comprises: one or more input-output (I/O) interfaces 141, 143, a control module 142, a memory module 144, and a power module 145. The I/O interface 141 is connected with the connecting interface unit 123 of the visualization processing module 12. Because the detailed architectures and features of the control module 142, memory module 144, and power module 145 are similar to those of a conventional IPC and thus are not described here. The I/O interface 143 of the IPC 14 is able to be connected with other external devices 15 or other display device 133. The external devices 15 can be ordinary or commonly used medical instruments, such as, but not limited to, surgical lamp machine, electrocardiographic machine, bipolar radio-frequency (RF) machine, cold light providing machine, power providing machine, etc.

In another embodiment of the present invention, the first and second electronic signals of the endoscope 11 are not transmitted via the cable 113 to the input unit 121 of visualization processing module 12. In this embodiment, the PCB 114 of the endoscope 11 further includes a wireless communicating module (not shown in figures), and the input unit 121 of visualization processing module 12 is communicating with the wireless communicating module of the endoscope 11 in a wireless manner, for example, but not limited to, Wi-Fi wireless@55/65 GHz bandwidth range. In addition, the Wi-Fi 1233 of the connecting interface unit 123 can also comply with the Wi-Fi wireless@55/65 GHz, such that the output visualization signal can be transmitted from the connecting interface unit 123 of visualization processing module 12 to the display devices 131, 132, smart-glasses and/or naked-eye 3D display for displaying by using Wi-Fi wireless@55/65 GHz bandwidth range. Such bandwidth is sufficient for transmitting the first and second electronic signals and the output visualization signal, no latency nor distortion would occur.

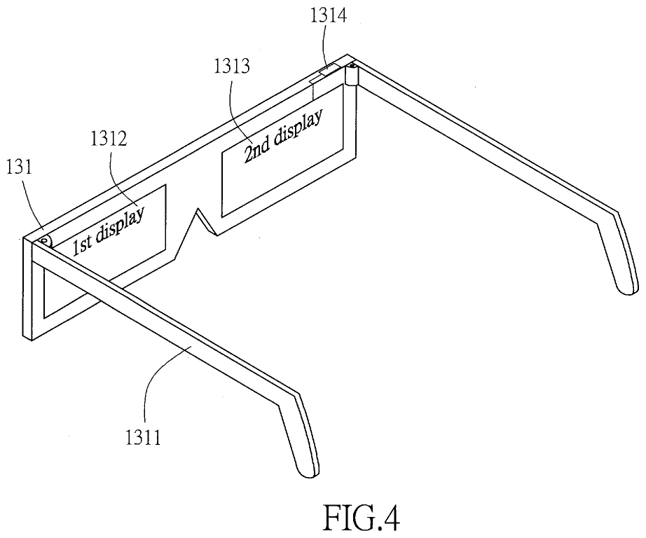

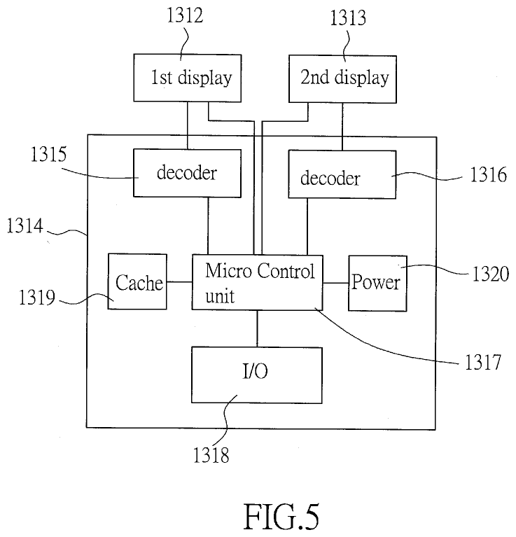

Please refer to FIG. 4 and FIG. 5, which respectively are a schematic view and a block diagram of the smart-glasses in accordance with the present invention. The smart-glasses (3D display device 131) is wearable by personnel and comprises: a glasses frame 1311, two display screens 1312, 1313 cooperating with the glasses, and a display module 1314 furnished in the glasses frame 1311. The smart-glasses (3D display device 131) comprises a left eye display screen 1312 and a right eye display screen 1313 for displaying the first image and the second image respectively in an Augmented Reality (AR) manner. One way to display the first image and the second image respectively in the AR manner on the two display screens 1312, 1313 of smart-glasses (3D display device 131) is to employ two transparent or semi-transparent glasses with liquid-crystal display (LCD) or organic light-emitting diode (OLED) panels on the left and right glasses of the smart-glasses (3D display device 131). The other way is to employ two mini-projectors for projecting the first image and the second image respectively on the left and right display screens 1312, 1313 of the smart-glasses (3D display device 131). The display module 1314 comprises a first decoder 1315, a second decoder 1316, a micro-control unit 1317, an I/O interface 1318, a cache memory 1319 and a power 1320. The I/O interface 1318 is to communicate with the connecting interface unit 123 of the visualization processing module 12 in order to receive the output visualization signal in either a wired or wireless manner. The micro control unit 1317 processes the output visualization signal and extracts the streaming of the first and second images contained the in the 3D video streaming of the output visualization signal. The extracted left image is then fed to the decoder 1315 and then displayed on the left eye display screen 1312. The extracted right image is fed to the other decoder 1316 and then displayed on the right eye display screen 1313. The cache 1319 is for temporary storing the images during the processing and decoding operations. In case the smart-glasses (3D display device 131) is connected with the visualization processing module 12 in a wired manner, the power 1320 includes a socket for accepting external electricity via the cord. In another case when the smart-glasses (3D display device 131) is connected with the visualization processing module 12 in a wireless manner, the power 1320 includes a battery and a charging module for providing electricity required.

In a preferred embodiment, the smart-glasses (3D display device 131) is configured wearable and expandable to include audio and speaker etc., such that the medical personnel 90 wearing the smart-glasses (3D display device 131) can talk to others by using the smart-glasses (3D display device 131) during the operation (surgery). Moreover, in addition to the visualization processing module 12, the smart-glasses (3D display device 131) can also be connected to the IPC 14 through wire or wireless mechanism.

In another preferred embodiment of the present invention, the smart-glasses (3D display device 131) further includes a helmet-like or hat-like structure (not shown in figures) furnished on top of the glasses frame 1311 for facilitating the comfortability and stability when the medical personnel 90 is wearing the smart-glasses (3D display device 131).

While the present invention has been particularly shown and described with reference to a preferred embodiment, it will be understood by those skilled in the art that various changes in form and detail may be without departing from the spirit and scope of the present invention.

25

What is claimed is:

1. An integrated endoscope and visualization system, comprising:
an endoscope, equipped with a first image capturing module, a second image capturing module, and a cable for electrically connecting said first image capturing module and said second image capturing module; said first image capturing module being capable of capturing a first image and transforming said first image into a first electronic signal; said second image capturing module being capable of capturing a second image and transforming said second image into a second electronic signal;
a visualization processing module, connected with the endoscope; said visualization processing module receiving said first electronic signal and said second electronic signal in order to generate an output visualization signal containing the first image and the second image; and
at least one 3D display device, for receiving said output visualization signal and displaying said first image and said second image in a 3D manner.

2. The integrated endoscope and visualization system of claim 1, wherein the first image capturing module and the second image capturing module are arranged in a side-by-side configuration and each comprises a lens set and a CMOS image sensor or CCD image sensor.

3. The integrated endoscope and visualization system of claim 1, wherein said 3D display device is one of the following: smart-glasses, naked-eye 3D display.

4. The integrated endoscope and visualization system of claim 3, wherein the smart-glasses comprises a left eye display and a right eye display for displaying the first image and the second image respectively in an Augmented Reality (AR) manner.

5. The integrated endoscope and visualization system of claim 1, wherein the visualization processing module comprises: an input unit connected with the cable for receiving said first electronic signal and said second electronic signal, a control unit connected with the input unit for processing received said first electronic signal and said second electronic signal in order to generate said output visualization signal, a memory unit for pre-storing an application for driving the control unit and caching said output visualization signal, and a connecting interface unit for communicating with at least the 3D display device.

6. The integrated endoscope and visualization system of claim 1, wherein the visualization processing module comprises: an input unit communicated with the endoscope in a wireless manner for receiving said first electronic signal and said second electronic signal, a control unit connected with the input unit for processing received said first electronic signal and said second electronic signal in order to generate said output visualization signal, a memory unit for pre-storing an application for driving the control unit and caching said output visualization signal, and a connecting interface unit for communicating with at least the 3D display device.

7. The integrated endoscope and visualization system of claim 5, wherein the visualization processing module communicates with the 3D display device by using one of the following interfaces: USB, HDMI, Wi-Fi, LAN, Airplay, Miracast, Bluetooth.

8. The integrated endoscope and visualization system of claim 1, further comprising an industrial personal computer (IPC) connected with the visualization processing module.

9. The integrated endoscope and visualization system of claim 1, wherein the endoscope is disposable and sterilizable, and can be detached from the visualization processing module.

10. An integrated endoscope and visualization system, comprising:
an endoscope, equipped with at least one image capturing module and a cable for electrically connecting said at least one image capturing module; said image capturing module being capable of capturing an image and transforming said image into an electronic signal;
a visualization processing module, connected with the endoscope; said visualization processing module receiving said electronic signal in order to generate an output visualization signal containing the image;
at least one display device, for receiving said output visualization signal and displaying said image; and
an industrial personal computer (IPC);
wherein, said visualization processing module is a modularized component and is connected to the IPC in a detachable manner.

11. The integrated endoscope and visualization system of claim 10, wherein the visualization processing module comprises: an input unit connected with the cable for receiving said electronic signal, a control unit connected with the input unit for processing received said electronic signal in order to generate said output visualization signal, a memory unit for pre-storing an application for driving the control unit and caching said output visualization signal, and a connecting interface unit for communicating with at least the display device.

12. The integrated endoscope and visualization system of claim 10, wherein the visualization processing module comprises: an input unit communicated with the endoscope in a wireless manner for receiving said first electronic signal and said second electronic signal, a control unit connected with the input unit for processing received said electronic signal in order to generate said output visualization signal, a memory unit for pre-storing an application for driving the control unit and caching said output visualization signal, and a connecting interface unit for communicating with at least the display device.

13. The integrated endoscope and visualization system of claim 11, wherein the visualization processing module communicates with the display device by using one of the following interfaces: USB, HDMI, Wi-Fi, LAN, Airplay, Miracast, Bluetooth.

14. The integrated endoscope and visualization system of claim 11, wherein:
said at least one image capturing module comprises a first image capturing module and a second image capturing module; said cable is electrically connecting to both said first image capturing module and said second image capturing module; said first image capturing module being capable of capturing a first image and transforming said first image into a first electronic signal; said second image capturing module being capable of capturing a second image and transforming said second image into a second electronic signal;
said visualization processing module receives said first electronic signal and said second electronic signal in order to generate said output visualization signal containing the first image and the second image;
said display device receives said output visualization signal and displays said first image and said second image in a 3D manner.

15. The integrated endoscope and visualization system of claim 14, wherein the first image capturing module and the second image capturing module are arranged in a side-by-side configuration and each comprises a lens set and a CMOS image sensor or CCD image sensor.

16. The integrated endoscope and visualization system of claim 14, wherein said 3D display device is one of the following: smart-glasses, naked-eye 3D display.

17. The integrated endoscope and visualization system of claim 16, wherein the smart-glasses comprises a left eye display and a right eye display for displaying the first image and the second image respectively in an Augmented Reality (AR) manner.

18. The integrated endoscope and visualization system of claim 10, wherein the endoscope is disposable and sterilizable, and can be detached from the visualization processing module.
